(19) United States
(12) Patent Application Publication
(10) Pub. No.: US 2011/0290694 A1
(43) Pub. Date: Dec. 1, 2011

(54) ORAL FILM DOSAGE FORM HAVING INDICIA THEREON

(75) Inventors: Richard C. Fuisz, Beverly Hills, CA (US); Madhu Hariharan, Munster, IN (US)

(73) Assignee: MONOSOL RX, LLC, Warren, NJ (US)

(21) Appl. No.: 12/788,760

(22) Filed: May 27, 2010

(51) Int. Cl. B65D 85/00; A61K 9/44; G06F 19/00; A61K 9/70

(52) U.S. Cl. 206/459.5; 424/443; 427/2.23; 702/84

(57) ABSTRACT

The present invention relates to rapidly dissolving edible film dosage form incorporating indicia. The indicia may correspond to an active ingredient that may be evenly distributed throughout the film. The indicia may be associated with at least one surface of the film composition and provide information to the consumer that is relevant to the edible film dosage form.

### FIELD OF THE INVENTION

The present invention relates to rapidly dissolving edible film dosage form incorporating indicia. The indicia may correspond to an active ingredient that may be evenly distributed throughout the films. The indicia may also provide information to the consumer regarding the edible film dosage forms.

### BACKGROUND OF THE INVENTION

Active ingredients, such as drugs or pharmaceuticals, may be prepared in a tablet form to allow for accurate and consistent dosing. However, this form of preparing and dispensing medications has many disadvantages including that a large proportion of adjuvants must be added to obtain a size able to be handled, that a larger medication form requires additional storage space, and that dispensing includes counting the tablets which has a tendency for inaccuracy. In addition, many persons, estimated to be as much as 28% of the population, have difficulty swallowing tablets. Although tablets may be broken into smaller pieces or even crushed as a means of overcoming swallowing difficulties, this is not a suitable solution for many tablet or pill forms. For example, crushing or destroying the tablet or pill form to facilitate ingestion, alone or in admixture with food, may also destroy the controlled release properties.

As an alternative to tablets and pills, films may be used to carry active ingredients such as drugs, pharmaceuticals, and the like. However, historically films and the process of making drug delivery systems therefrom have suffered from a number of unfavorable characteristics that have not allowed them to be used in practice.

It has been known to use printing techniques to place indicia on small, hard articles, such as pharmaceutical capsules, tablets, and confectionery products. These indicia may be in the form of a trademark or lot number. For example, U.S. Pat. No. 4,528,904, discloses using a printing apparatus and various printing machines to print indicia on such articles; U.S. Pat. No. 4,905,589 discloses using an apparatus for inkjet marking such articles; and U.S. Patent Appl. No. 2002/0114863 discloses a method and apparatus for printing indicia on a confectionery product.

However, the prior art does not specify applying an indicia to a dissolvable film dosage form and using the indicia to indicate the presence of multiple active components or other information regarding the film dosage form. Therefore, there is a need for an edible film dosage form with an indicia, whereby the indicia is representative of the content of an active component in the film. Further, there is a need for methods of indicating the presence of an active in an edible film dosage form.

### BRIEF DESCRIPTION OF THE DRAWINGS

FIG. 1 is a representation of a sheet of dissolvable thin film and indicia being applied thereon via a gravure roll.

FIG. 2 is a representation of different types of indicia on pieces of dissolvable thin film.

FIG. 3 is a representation of a dissolvable thin film with indicia that includes a bar code.

FIG. 4 is a representation of low, medium and high density barcodes.

### SUMMARY OF THE INVENTION

The present invention relates to an edible film dosage form. The edible film dosage form includes indicia which includes information, such as information regarding the content or use of the edible film dosage forms.

In some embodiments, there is provided an edible film dosage form including: (a) a film composition including a film-forming polymer and at least one active component, where the film composition has at least one surface; and (b) at least one indicia associated with at least one surface of the film composition, where the at least one indicia includes information.

In some embodiments, there is provided an edible film dosage form including: (a) a film composition including a film-forming polymer and at least one active component, where the film composition has at least one surface and (b) at least one indicia associated with the at least one surface of the film composition, where the at least one indicia includes a monitoring agent.

In other embodiments, there is provided an edible film dosage form including: (a) a film composition including a film-forming polymer and at least one active component; where the film composition has at least one surface and (b) at least one indicia associated with the at least one surface of the film composition, where the at least one indicia includes multiple levels of information.

In some embodiments, there is provided a pharmaceutical film dosage form including: (a) a film composition including at least one water-soluble polymer and at least one pharmaceutical active component; where the film composition has at least one surface and (b) at least one indicia associated with at least one surface of the film composition, where the at least one indicia includes information.

Some embodiments provide a packaged edible film dosage form including (a) at least one film composition including: a film-forming polymer and at least one active component, the at least one film composition being enclosed within a sealed pouch, where the sealed pouch includes a top layer, a bottom layer, an inner cavity and has at least one surface and (b) at least one indicia associated with the at least one surface of the sealed pouch, where the at least one indicia includes at least one level of information.

In some embodiments, there is a method for indicating the presence of an active component in a film dosage form including the steps of: (a) providing a film composition including at least one film-forming polymer, and at least one active component, the film composition having at least one surface and (b) applying at least one indicia to the at least one surface of the film composition, where the at least one indicia corresponds to at least one active present in the film composition thereby indicating the presence of the at least one active.

In some embodiments, there is provided a method of applying indicia on an edible film composition including: (a) providing a sheet of edible film composition including a film-forming polymer, and at least one active component, where the sheet of edible film composition has at least one surface and (b) applying indicia to the surface of the sheet of edible film composition, where the indicia includes information corresponding to the edible film composition.

In some embodiments, there is provided a method of providing information to a consumer with respect to an edible film product including: (a) providing a sheet of edible film, the sheet having at least one surface for displaying indicia and (b) applying indicia selected from a barcode, a pattern, an image, a color, a shape or combinations thereof, where the indicia provides information regarding the content or use of the edible film product.

In some embodiments, there is provided a method of providing information to a consumer with respect to a packaged edible film dosage form including: providing a sheet of edible film, the sheet having at least one surface for displaying indicia; dividing the sheet of edible film into individualized dosage forms; enclosing the individualized dosage forms in a sealed pouch; and applying indicia selected from a barcode, a pattern, an image, a color, a shape or combinations thereof, where the indicia provides information regarding the content or use of the product.

Some embodiments provide a method of monitoring production of packaged edible film dosage forms including: providing a packaged edible film dosage form including a sheet of edible film divided into individualized dosage forms, the sheet having at least one surface for displaying indicia, a sealed pouch and at least one indicia, the indicia including information; entering data into an input device to represent a quality range; evaluating the information with analysis unit; generating a comparison of the information and the data; analyzing the comparison; and utilizing the comparison to verify accuracy of the production of the packaged edible film dosage forms.

In some embodiments, there is provided a method of testing a packaged edible film dosage form for quality control purposes including: irradiating a sample packaged edible film dosage form to establish a reference signal; measuring the value of a packaged edible film dosage form as it passes through a radiation device; comparing the value against the reference signal to create an output signal; and using the output signal to classify the packaged edible film dosage form for quality control purposes.

In some embodiments, there is provided a method of incorporating multiple levels of information into an edible film composition including: providing a film composition including at least one film-forming polymer, and at least one active component, the film composition having at least one surface; applying at least one indicia to the at least one surface of the film composition, where the at least one indicia provides a level of information selected from a primary level of information, a secondary level of information, a tertiary level of information, and combinations thereof.

### DETAILED DESCRIPTION OF THE INVENTION

The edible film dosage forms of the present invention are made, in general, by applying indicia to a film composition. The packaged edible film dosage forms of the present invention are made, in general, by applying indicia to a pouch which includes a film composition.

In one aspect of the invention, there is provided an edible film dosage form including a film composition with at least one surface, where the film composition includes a film-forming polymer, at least one active component, and at least one indicia associated with the at least one surface, where the at least one indicia provides information.

In another aspect of the invention, there is provided a pharmaceutical film dosage form including a film composition, including at least one water-soluble polymer, at least one pharmaceutical active; and at least one indicia associated with at least one surface of the film composition.

### Indicia

The term “indicia” is used herein to denote markings of any kind. It refers to any polychromatic or monochromatic design, picture, text, pattern or other symbology which may be incorporated in the present invention. Useful indicia may be selected from barcodes, patterns, stripes, straight stripes, wavy stripes, letters, numbers, dots, spots, concentric circles, lines, specks, geometric shapes, complex shapes, alphanumerics, and combinations thereof. Desirably, the indicia may be in the form of a barcode.

The term “information” is used herein to denote communications of any kind. It refers to any facts or circumstances which may relate to the present invention. The information may be fixed or variable and include a communication selected from barcodes, ingredients, expiration dates, lack of ingredients, side effects, a product name, a manufacturer, a dosage amount, a lot number, a batch number, a date, a time, and combinations thereof.

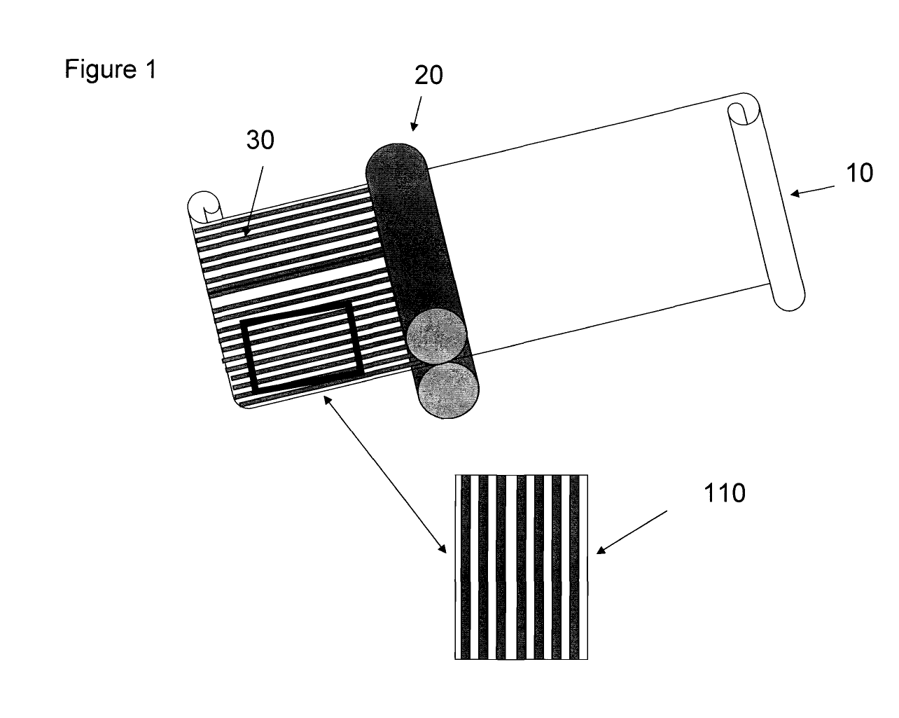

Furthermore, the indicia may include particles or particulates that may be added to the film-forming composition or matrix after the composition or matrix is cast into a film. For example, particles may be added to the film prior to the drying of the film. Particles may be controllably metered to the film and disposed onto the film through a suitable technique, such as through the use of a doctor blade (not shown) which is a device which marginally or softly touches the surface of the film and controllably disposes the particles onto the film surface. Other suitable, but non-limiting, techniques include the use of an additional roller to place the particles on the film surface, spraying the particles onto the film surface, and the like. For example, as shown in FIG. 1, edible film dosage form 110 is formed after applying indicia 30 via gravure role 20 to the surface of film sheet 10. The indicia or particles may be placed on either or both of the opposed film surfaces, i.e., the top and/or bottom film surfaces. Desirably, the indicia or particles are securably disposed onto the film, such as being embedded into the film. Moreover, such particles or indicia are desirably not fully encased or fully embedded into the film, but remain exposed to the surface of the film, such as in the case where the particles are partially embedded or partially encased.

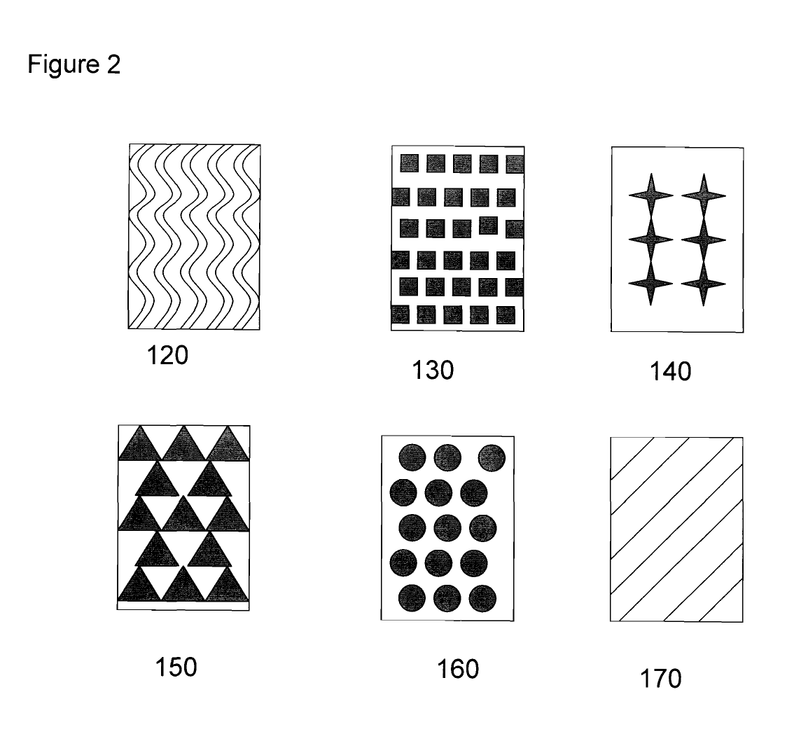

Examples of patterns that may be used as indicia are shown in FIG. 2. In some embodiments of the invention, the edible film dosage form may include one indicia. In other embodiments, the edible film dosage form may include at least two indicia. For example, the indicia may include wavy lines 120, squares 130, stars 140, triangles 150, circles 160 or diagonal lines 170. The first indicia may be selected from barcodes, patterns, straight stripes, wavy stripes, letters, numbers, dots, spots, specks, speckled stripes, geometric shapes such as polygons, complex shapes, pictures, and combinations thereof. The second indicia may be selected from barcodes, patterns, straight stripes, wavy stripes, letters, numbers, dots, spots, specks, geometric shapes such as polygons, complex shapes, pictures, and combinations thereof. The first indicia may be the same or different from the second indicia.

### Bar Codes

A bar code is a machine-readable representation of data. The use of bar coding to identify the contents of a product is widespread in the medical industry. For example, bar code identification systems are used in hospitals to track its inventory of pharmaceutical products electronically. Bar codes are also used in automated agent compounding systems to properly mix the correct and proper amounts of medical and therapeutic agents. More important, bar codes also allow hospitals to monitor its medications or other therapeutic fluids that are targeted for infusion into its patients by marking the same with fixed information such as product code names or numbers.

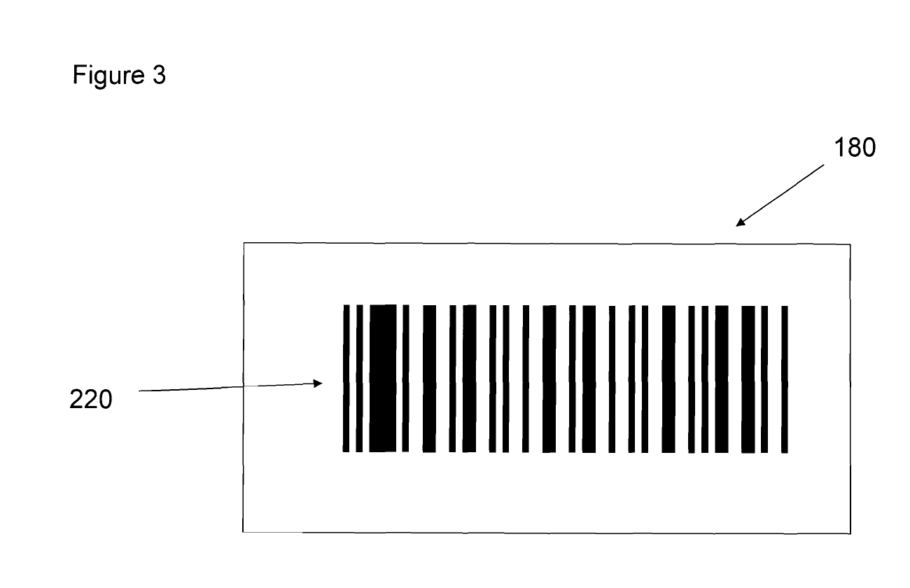

Traditionally, a two-color system consisting of black lines on a white background was implemented in bar coding systems. As such, when a bar code reader would read the bar code, the black lines would absorb the reader's laser light while the white spaces would reflect the reader's laser light back to the reader where the reflected information was translated into its corresponding analog counterpart. An example of this is found in FIG. 3, wherein the package 180 includes a bar code 220.

Bar codes are currently used in many industrial fields as a means for acquiring the identification information about products in an easy and secure manner. For example, in various wholesale/retail shops, products are identified from the bar codes attached thereon and the corresponding prices (stored in memory in advance) are read from a database to be displayed on a display unit.

Barcodes are useful forms of indicia because a vast amount of information may be incorporated into such a small space. The Internet, as well as any other global electronic or computer network, provides access to information relating to a company and their products, all of which may be incorporated into the barcode.

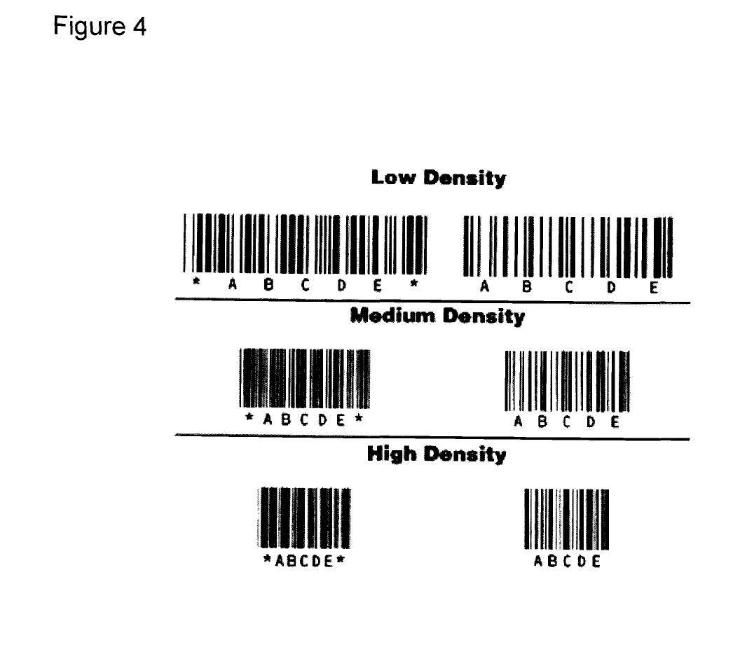

In some embodiments, the bar code may incorporate the Uniform Product Code (hereinafter “UPC), as shown in FIG. 4. The UPC system was developed for product identification in the United States and is administered by a Uniform Code Council. As such, a manufacturer interested in utilizing the UPC system registers with the Uniform Code Council and receives a unique manufacturer code. The UPC of a product is a combination of the manufacturer's code and a product code assigned by the manufacturer. To facilitate product identification, the UPC is encrypted as a barcode and placed on the product, where it is machine readable, such as by a scanner at a supermarket checkout counter, and used to digitally identify the product. Once the product is so identified, the digital barcode information read therefrom may be communicated to many different destinations for accounting and inventory purposes and to various databases for recording and archiving.

The UPC system has been adapted for use with computers and networks, such as the Internet, in various ways. For example, U.S. Pat. No. 5,913,210, to C. C. CALL, which disclosure is incorporated herein by reference, discloses a system involving the storing of a plurality of UPCs in respective computers with web addresses (URLs) and storing cross-references of the UPCs and URLs in another computer (URL) which can be accessed by further networked computers using the UPCs to find the respective URLs. Also, U.S. Pat. No. 5,804,803, to B. J. CRAGUN ET AL, which disclosure is incorporated herein by reference, discloses the retrieval of a document by a client computer system using a scanned UPC to create a URL location in a first server, from UPC and customer data retrieved from a second server, which URL may be used to obtain the document from the first server. Further, U.S. Pat. No. 5,791,991, to M. E. SMALL, which disclosure is incorporated herein by reference, discloses an interactive product promotion system which enables the selection of coupons to be downloaded from the Internet, and then UPCs placed thereon to be scanned to the Internet for rebates.

In some embodiments, barcodes such as those described in U.S. Pat. No. 7,059,526 and U.S. Pat. No. 7,061,831, which disclosure is incorporated herein by reference, may be used. For example, any machine readable code may be used. Useful barcodes include alpha-numeric characters, high or low density barcodes, 2-dimensional micro barcodes, 3 dimensional barcodes, a UPC/EAN/JAN, Code 128, Code 39, Code Inerleaved 2 of 5, EAN 128, Codabar, PDF417, UPC-A, UPC-E, EAN 8, EAN 13, UPC/EAN 128 or any combinations thereof. Each of the codes must be sized appropriately so as to be suitable for use on the dosage forms in accordance with the invention.

Barcodes such as those disclosed in U.S. Pat. No. 3,991,300 may be used. For examples, these barcodes may have various widths which are multi-colored in order to designate the required binary information.

High or low density barcodes, such as those described in U.S. Pat. No. 5,311,001, which disclosure is incorporated herein by reference, may be used. A high density barcode may include a variable number of component symbols or codes per row and have a variable number of rows. The codes in the alternating rows may be the same or different. Low density barcodes typically have larger widths and require more space to print. Examples of high, medium and low density bar codes appear in FIG. 4.

Two or three dimensional barcodes, such as those described in U.S. Pat. No. 5,414,250, which disclosure is incorporated herein by reference, may be used.

The barcode may correspond to the type as disclosed in U.S. Pat. No. 4,864,112. For example, in some embodiments the barcode may have a plurality of parallel bar elements, each of which has a width having some ratio relationship of bar element width to a reference width, the width of each bar element indicating a code of the encoded information, the bar code being divided into at least three groups, each having a plurality of the bar elements and a ratio associated with these widths, a third group being provided between a first and a second group, the ratio of the second group being greater than the ratio of the third group.

The barcode may include information relating to ingredients, lack of ingredients, side effects, a product name, a manufacturer, a dosage amount, a lot number, a batch number, a date, a time, and combinations thereof. In some embodiments, the barcode may be printed on the surface of the embodiment. In some embodiments, the barcode is representative of the content of one or more active components. In other embodiments, the barcode may represent the relative content of two or more active components.

In some embodiments, the barcode may include a monitoring agent. Desirably the monitoring agent may be used for quality or inventory control of the edible film dosage forms.

The information may be encoded in the barcode.

### Communications

The information may include a communication selected from barcodes, ingredients, lack of ingredients, side effects, expiration dates, a product name, a manufacturer, a dosage amount, a lot number, a batch number, a date, a time, and combinations thereof. Some embodiments may have the indicia printed on the surface. In some embodiments, the information is representative of the content of one or more active components. In other embodiments, the information may represent the relative content of two or more active components.

In some embodiments, the indicia may include fixed information, such as a product's name, code number, manufacturer, National Drug Code Number, label copy data required by the Federal Food & Drug Administration (FDA), or data required by the Health Industry Bar Code Council, now known as the Health Industry Business Communications Council (HIBCC), and the like, that remains unchanged for a period of time.

In some embodiments, the indicia may include variable information, such as a product's lot number, batch number, expiration date, serial number, production time, price, inventory control data, and concentration, whereby the information may change during a period of time.

In some embodiments, the information is representative of the content of one or more active components. In other embodiments, the information may represent the relative content of two or more active components.

In some embodiments, the information may include any of those described in U.S. Pat. No. 7,111,780, which disclosure is incorporated herein by reference, may be used. For example, the information may be a common drug identifier, such as a National Drug Code (NDC). The NDC number is a 10-digit number typically containing three (3) segments or fields: the manufacturer or distributor code field, the product code field, and the package code field and is issued for each drug in the United States. The manufacturer or distributor code is assigned by the Food and Drug Administration (FDA), whereas the product and package codes are assigned by manufacturers or distributors.

The manufacturer or distributor code identifies the entity that manufactured or distributed the drug. The product code represents the drug name and strength. The package code represents the quantity (count, weight, mass or volume) in the stock container as delivered from the manufacturer or distributor. The three field representation may result in numerous NDC numbers being assigned to the same drug. Although numerous NDC numbers assigned to the same drug may be inefficient for drug identification, the NDC numbering scheme facilitates inventory management processes.

The NDC number may be represented in one of three different formats typically denoted as 4-4-2, 5-3-2 and 5-4-1. The first of the three segments represents the manufacturer or distributor code and may be either 4 or 5 digits. When the manufacturer code is 4 digits, the product code field will be 4 digits and the package code field will be 2 digits (i.e., the 4-4-2 format). When the manufacturer code is 5 digits, the manufacturer/distributor has an option of assigning either a 3 or 4 digit product code field followed by a 2 or 1 digit package code field (i.e., the 5-3-2 and 5-4-1 formats, respectively). The NDC number may be encoded on the films and products of the present invention as a barcode using the symbology reserved for NDC numbers as defined by the Uniform Code Counsel (UCC). In addition to the NDC system used in the United States, foreign countries may use different drug identifiers.

Other identification systems may be assigned (for example, by a manufacturer, supplier, pharmacy and the like) to identify a medical item. For example, a general product index number (GPI), a general product code (GPC), and/or an internal reference code (IEN) may be assigned to a medical item by a manufacturer.

In some embodiments, the information may be visually perceptible.

### Hidden Information

In some embodiments, the barcode or other indicia may be too small and/or complex for the unaided eye to discern differences between different edible film dosage forms. The term “hidden information” is meant to denote any indicia that may be invisible to the naked eye. The invisible indicia may be decoded with a suitable reading system, such as those described in U.S. Pat. No. 7,059,526 and U.S. Pat. No. 5,331,140, the entire disclosures of which are incorporated herein by reference.

In some embodiments, the information may include a communication which is invisible to the naked eye. In some embodiments, the communication may include hidden information such as that described in U.S. Pat. No. 6,427,921 and U.S. Pat. No. 5,957,458, the entire disclosures of which are incorporated herein by reference. For example, such hidden information may include barcodes or patterns which are extremely fine so that the barcodes or patterns are difficult to be reproduced with an image scanner or a photocopier. Such hidden information may provide valuable communications to a user or manufacturer once it has been decoded.

### Monitoring Agents

In some embodiments, the indicia may include a monitoring agent. The term “monitoring agent” as used herein denotes any mark useful for control, inspection or verification purposes which may relate to the present invention. The monitoring agent may include a substance which makes the present invention particularly suitable for providing a user with a visual quality control indication on the dosage form. Such a substance may visibly alter the indicia if the dosage form were exposed to adverse humidity or temperature conditions. Alternatively, the monitoring agent may include information particularly suitable for providing a manufacturer with information relating to quality control of the processing or manufacturing of the dosage form. Desirably the monitoring agent may be used for quality or inventory control of the edible film dosage forms.

Quality control of the edible film dosage forms may include the expiration date, type, purity, amount of ingredients, and/or defects in the film.

In some embodiments, it is desirable to use color to provide information to a consumer such as when to take a dose, i.e., the time of day or day of the week to take the dose.

In some embodiments, the edible film dosage may include two indicia, the first indicia including a first coloring agent and the second indicia including a second coloring agent, where the first coloring agent is different from the second coloring agent. In other embodiments, the first coloring agent may contrast with the second coloring agent. Examples of useful coloring agents include any of those as described above.

In some embodiments, the film composition may also include an excipient in addition to the indicia. The excipient may include a first coloring agent and the indicia may include a second coloring agent, where the first coloring agent is different from or contrasts the second coloring agent.

In some embodiments, the information may provide a primary indication to a consumer. In other embodiments, the information may provide a level of information to a consumer selected from a primary indication, secondary indication, tertiary indication and combinations thereof.

In some embodiments, the indicia is representative of the content of one or more active components. In other embodiments, the indicia may be representative of the relative content of two or more active components.

In some embodiments, the indicia may represent the ratio of a combination of active components which are present in the edible film dosage form.

In other embodiments, the indicia may include multiple levels of information. In some embodiments, the indicia may include a primary level of information which includes a direct meaning. The direct meaning immediately conveys the information to a consumer, doctor, pharmacist, manufacturer, health care administrator or the like. Examples of useful primary levels of information may include ingredients, lack of ingredients, side effects, product name, product manufacturer, dosage amount, lot number, batch number, date, time, the time of day or week the edible film dosage form should be administered into the oral cavity and combinations thereof. In other embodiments, the indicia may include a secondary level of information which includes an indirect meaning. An indirect meaning must be deciphered and is not immediately conveyed to the consumer, doctor, pharmacist, manufacturer, administrator and the like. Examples of useful secondary levels of information may include a code, a barcode, an infrared colorant, and the like. In some embodiments the decipherable information may indicate to a consumer, manufacturer or pharmacist that the edible film dosage form contains a controlled substance.

### Molecular Tag

The indicia may include a molecular tag. A molecular tag, such as those described in U.S. Patent Application No. 2004/0137458, which disclosure is incorporated herein by reference, may be used. For example, such a tag may be useful for a monitoring, detecting or tracing substances and may include a single or double-stranded nucleic acid region with two ends being capable of pairing with a complementary nucleotide sequence, and at least one marker sequence having a number of non-complementary nucleotides sufficient to minimize or prevent the formation of secondary structure within the marker under normal conditions of use.

### Luminescent Materials

The indicia may include luminescent materials such as bioluminescent or chemiluminescent materials. In some embodiments, bioluminescent materials such as those described in U.S. Pat. No. 6,152,358, which disclosure is incorporated herein by reference, may be used.

For example, luciferase genes, which have been cloned and exploited as reporter genes in numerous assays, for many purposes, may be used. As such, different luciferase systems having different specific requirements may be used to detect and quantify a variety of substances. Any bioluminescent material may be used, including those based on firefly luciferase and aequorin, a purified jellyfish photoprotein. Many bioluminescent materials have been studied and well-characterized and are commercially available [e.g., firefly luciferase is available from Sigma, St. Louis, Mo., and Boehringer Mannheim Biochemicals, Indianapolis, Ind.; recombinantly produced firefly luciferase and other reagents based on this gene or for use with this protein are available from Promega Corporation, Madison, Wis.; the aequorin photoprotein luciferase from jellyfish and luciferase from Renilla are commercially available from Sealite Sciences, Bogart, Ga.; coelenterazine, the naturally-occurring substrate for these luciferases, is available from Molecular Probes, Eugene, Oreg.]. These luciferases and related reagents are used as reagents for diagnostics, quality control, environmental testing and other such analyses.

Examples of luminescent materials may include a bioluminescent generating system selected from: Aequorea, Vargula, Renilla, Obelin, Porichthys, Odontosyllis, Aristostomias, Oplophorus, Gaussia, firefly, bacterial, Mnemiopsis, Beroe Gonadostomias, Gaussia, Halisturia, Vampire squid, Glyphus, Mycotophid, Vinciguerria, Howella, Florenciella, Chaudiodus, Melanocostus Sea Pens, mollusk, mushroom, fish, insect, ctenophore and annelid systems. The luminescent material may be embedded in the dosage form, on the surface of the dosage form, on a surface associated with the dosage form, or on a pouch which may include the dosage form.

### Holographs

The use of holograms or holographic images has been used to record images on pharmaceutical products. For example, U.S. Pat. No. 4,668,523 to Begleiter discloses a system for applying a high resolution diffraction gratings to a food product to produce edible holograms. In some embodiments, the indicia may include a holograph such as those described in U.S. Pat. No. 7,083,805, which disclosure is incorporated herein by reference. Such indicia may be incorporated on the film products of the present invention with a technique similar to those described in “Edible Holography: The application of holographic techniques to food processing”, SPIE, Vol. 1461, “Practical Holography V” (1991) at pages 102-109, which discusses the use of a punch die to compress a powder into a tablet while simultaneously using a metal die plate to impress a microrelief as the powder becomes a solid core in a tablet press. The indicia may convey information such as visual holographic images and effects, such as rainbow-like color patterns, pictures, and changes in color or location of pictures or parts of pictures with a change in viewing angle.

A hologram for use in the films of the present invention may include a layer of material that is capable of receiving and retaining a high resolution microrelief that can convey information. The material may be thermoformable and formed from an aqueous solution of a thermoformable material selected from gelatin, hydroxypropylmethylcellulose (HPMC), hydroxyproplycellulose (HPC), modified food starches, waxes, vegetable gums and combinations thereof. The material may also include the following plasticizers, such as hygroscopic plasticizers; colorants; and oils and waxes, such as paraffin or carnuba.

A method for producing a hologram on a dosage form includes the steps of: (a) coating the dosage form with a layer of thermoformable material that can receive and retain a holographic diffraction pattern; (b) providing a plate having a holographic diffraction pattern formed on at least a portion of a first surface thereof (c) transporting the coated cores to a position opposite the first plate surface; (d) heating at least one of the plate and the coated layer during or prior to the time when they are in the opposed relationship; (e) pressing the first plate surface into the coated layer to replicate the holographic diffraction pattern in the coated layer; (f) cooling the coated layer thus replicated; and (g) demolding the first plate surface from the coated layer.

### Application of the Indicia

The present invention also is directed to methods of making the edible multi-layer films. In particular, a first water-soluble film layer, as described above, is provided. One or more additional water-soluble film layers, which are the same as or different from the first, are positioned in at least partial face-to-face engagement with the first layer. The first and additional layers are sealed together at the face-to-face engagement. Desirably, a heat seal is formed, optionally with the use of pressure.

When the layers are in full face-to-face engagement, they may be fully laminated together to form a multi-layer film.

When the layers are in partial face-to-face engagement at the perimeters of the film layers, the layers may be perimetrically sealed together, and in addition may also have sealed sections internal to the perimeter, such as in the case of a multi-pocket embodiment. A pocket is thereby defined between the film layers. In some embodiments, the indicia may include an active that is applied to the first film layer prior to positioning the additional film layer on the first layer. In multi-pocket embodiments, different actives may be contained in the different pockets. These actives may dissolve at different times or conditions, e.g., different temperatures or pH.

The indicia may include an active that may be in the form of a powder. The powder may be sprinkled onto the first film layer or into a coating that may be applied by spraying or brushing thereon. Once the additional film layer is added, the layers are sealed together, thereby housing the active in the pocket between the layers. Additional film layers may then be added in a similar manner.

More specifically, the first film layer may be provided over a mold, which has a plurality of cavities in the desired shape of the final film product. A vacuum may be applied to the first film layer positioned in the cavities. Subsequently, the active component may be added to the cavities, and then the additional film layer may be added to the top. Heat and/or pressure may be applied to seal the film layers together at the desired location.

Alternatively, a water-soluble film, as described above, is provided. The film is then folded over upon itself, thereby creating two film layers. The film layers are then sealed together at their at least partial face-to-face engagement. When the face-to-face engagement is at the perimeters of the layers, the film is thereby sealed on three sides.

In some embodiments, the indicia may include edible ink. Desirably, the ink is nontoxic so as to not render the edible film dosage form essentially inedible. A variety of known edible inks are appropriate, such as those with a shellac base in ethyl alcohol. For example, one typical variety of edible ink includes shellac, ethyl alcohol, isopropyl alcohol, n-butyl alcohol, propylene glycol, ammonium hydroxide, and FD&C Blue. In some embodiments, edible inks such as those described in U.S. Pat. No. 6,623,553, which disclosure is incorporated herein by reference, may be used. For example, such inks may include water, at least one sweetener, at least one emulsifier, and a humectant. In some embodiments, edible ink such as those described in U.S. Pat. No. 7,128,938, which disclosure is incorporated herein by reference, may be used. For example, such inks may include isopropyl or ethyl alcohol, glycerin, FD&C food color, and distilled water.

In some embodiments, the indicia may include edible ink and an active. Examples of useful actives may include any of those listed above.

In some embodiments, the at least one indicia may be printed on at least one surface of the film composition. The indicia may be printed to be read either in the horizontal direction, vertical direction or at an angle. When more than one indicia is included, the first indicia may be printed in the same or a different position than the second indicia or any subsequent indicia. In other embodiments, the at least one indicia may be embedded in the at least one surface of the film composition. In some embodiments, the indicia may be in contact with at least one surface of the film composition. In other embodiments, the indicia may be associated with at least one surface of a sealed pouch which surrounds the film dosage form and includes at least one level of information.

In some embodiments, the at least one indicia is printed on the film dosage form using a printing device selected from rotogravure, lift up, ink jet, and bubble jet. In some embodiments, a rotogravure printing device such as those described in U.S. Pat. No. 4,528,904 which disclosure is incorporated herein by reference, may be used.

In some embodiments, a lift up printing device such as those described in U.S. Patent Publication No. 2002/0114863, which disclosure is incorporated herein by reference, may be used.

In some embodiments, an ink-jet technique such as those described in U.S. Pat. No. 6,994,254, which disclosure is incorporated herein by reference, may be used. Useful ink-jet printers may include an ink cartridge with edible ink. The ink cartridge may be UV based and contain infrared dyes.

In some embodiments, the indicia may be photocopied onto the edible film dosage form. For example, a printing technique such as those described in U.S. Pat. No. 6,582,742, which disclosure is incorporated herein by reference, may be used.

In some embodiments, a bubble-jet technique such as those described in European Patent No. EP 0876107 B1, which disclosure is incorporated herein by reference, may be used. For example, the shape or form of the indicia may be stored in a computer system in an encrypted manner. The computer system may then convert the image to a decipherable appearance upon incorporation of the bubble-jet printer head assembly. The bubble-jet printer head assembly may cause a colorant or otherwise edible ink to release from the printer head assembly upon receipt of the indicia from the computer system and then apply the indicia to the edible film dosage form.

In some embodiments, the indicia may cover the entire portion of at least one surface of the film composition. In some embodiments, the absence of indicia may be indicative of the contents of the film. For example, the indicia may be distinguished from those films that are completely covered with indicia. In addition, some films may include a mark that may only be visible under a specific light source, or if covered by a faux colored substance, such as a specifically colored cellophane packaging.

The indicia may be printed on the edible film with any type of non-harmful, biocompatible substance when digested. The printed indicia may be an edible substance, such as those disclosed in U.S. Pat. No. 7,059,526, which disclosure is incorporated herein by reference. For example, the edible substance may include a protein base material such as gelatin, collagen, or keratin. The edible substance may also include other materials such as waxes, polymeric materials, sugar based substrates or water-soluble plastics including polyvinyl alcohol. The barcode may also be printed on the edible film with certain plastics, including MYLAR or an ethylene-vinyl alcohol sheet or film.

The indicia may be printed on the edible film using any of the techniques known in the art, such as those described in U.S. Pat. No. 7,059,526, U.S. Pat. No. 5,006,362 or U.S. Pat. No. 4,889,367, each of which is disclosed herein by reference.

In some embodiments, the printed indicia may include an active. The printed indicia may convey the name of the active to the consumer.

### Film-Forming Polymers

The polymer may be water-soluble, water-swellable, water-insoluble, or a combination of one or more either water-soluble, water-swellable or water-insoluble polymers. The polymer may include cellulose or a cellulose derivative. Specific examples of useful water-soluble polymers include, but are not limited to, polyethylene oxide (PEO), pullulan, hydroxypropylmethyl cellulose (HMPC), hydroxyethyl cellulose, hydroxypropyl cellulose (HPC), polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. In some embodiments, combinations of PEO and a cellulosic polymer, such as hydroxypropyl cellulose, are employed. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof.

As used herein the phrase “water-soluble polymer” and variants thereof refer to a polymer that is at least partially soluble in water, and, desirably, fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. Desirably, the water-soluble polymers are water-soluble or water-swellable having at least 20 percent by weight water uptake. Water-swellable polymers having a 25 or greater percent by weight water uptake are also useful. Films or dosage forms of the present invention formed from such water-soluble polymers are desirably sufficiently water-soluble to be dissolvable upon contact with bodily fluids.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanones, polyoxalates, poly(*-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of *-amino acids, copolymers of *-amino acids and caproic acid, copolymers of *-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del., and are generically identified as a “lactide/glycolide co-polymer” containing “propanoic acid, 2-hydroxy-polymer with hydroxy-polymer with hydroxyacetic acid.” Four such polymers include the following: lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers to provide a desired viscosity of the mixture prior to drying. For example, if the active or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, desirably from about 800 cps to about 60,000 cps, and most desirably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling, which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity-increasing component. Components that will increase the viscosity or stabilize the emulsion/suspension include higher molecular weight polymers and polysaccharides and gums, which include, without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

### Actives

When an active is introduced to the film, the amount of active per unit area is determined by the uniform distribution of the film. For example, when the films are cut into individual dosage forms, the amount of the active in the dosage form can be known with a great deal of accuracy. This is achieved because the amount of the active in a given area is substantially identical to the amount of active in an area of the same dimensions in another part of the film. The accuracy in dosage is particularly advantageous when the active is a medicament, i.e., a drug.

The active components that may be incorporated into the films of the present invention include, without limitation, pharmaceutical and cosmetic actives, drugs, medicaments, antigens or allergens such as ragweed pollen, spores, microorganisms including bacteria, seeds, mouthwash components such as chlorates or chlorites, flavors, fragrances, enzymes, preservatives, sweetening agents, colorants, spices, vitamins and combinations thereof.

A wide variety of medicaments, bioactive active substances and pharmaceutical compositions may be included in the dosage forms of the present invention. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (commercially available as Oxycontin®); ibuprofen (commercially available as Motrin®, Advil®, Motrin Children's®, Motrin IB®, Advil Children's®, Motrin Infants'®, Motrin Junior®, Ibu-2®, Proprinal®, Ibu-200®, Midol Cramp Formula®, Bufen®, Motrin Migraine Pain®, Addaprin® and Haltran®), aspirin (commercially available as Empirin®, Ecotrin®, Genuine Bayer®, and Halfprin®), acetaminophen (commercially available as Silapap Infant's®, Silapap Children's®, Tylenol®, Tylenol Children's®, Tylenol Extra Strength®, Tylenol Infants' Original®, Tylenol Infants'®, Tylenol Arthritis®, T-Painol®, Q-Pap®, Cetafen®, Dolono®, Tycolene®, APAP® and Aminofen®), and combinations thereof that may optionally include caffeine. Other pain relieving agents may be used in the present invention, including meperidine hydrochloride (commercially available as Demerol®), hydromorphone hydrochloride (commercially available as Dilaudid®), propoxyphene napsylate and acetaminophen (commercially available as Darvocet-N®), Fentanyl (commercially available as Duragesic® and Fentora®), sodium hyaluronate (commercially available as Euflexxa®), adalimumab (commercially available as Humira®), sumatriptan succinate (commercially available as Imitrex®), fentanyl iontophoretic (commercially available as Ionsys®), orphenadrine citrate (commercially available as Norgesic®), magnesium salicylate tetrahydrate (commercially available as Novasal®), oxymorphone hydrochloride (commercially available as Opana ER®), methocarbamol (commercially available as Robaxin®), carisoprodol (commercially available as Soma®), tramadol hydrochloride (commercially available as Ultracet® and Ultram®), morphine sulfate (commercially available as MS Contin®), metaxalone (commercially available as Skelaxin®), oxycodone hydrochloride (commercially available as OxyContin®), acetaminophen/oxycodone hydrochloride (commercially available as Percocet®), oxycodone/aspirin (commercially available as Percodan®), hydrocodone bitartrate/acetaminophen (commercially available as Vicodin®), hydrocodone bitartrate/ibuprofen (commercially available as Vicoprofen®), nepafenac (commercially available as Nevanac®), and pregabalin (commercially available as Lyrica®).

The present invention may further include agents such as NSAIDs, including etodolac (commercially available as Lodine®), ketorolac tromethamine (commercially available as Acular®), naproxen sodium (commercially available as Anaprox®, Naprosyn®), flurbiprofen (commercially available as Ansaid®), diclofenac sodium/misoprostol (commercially available as Arthrotec®), celecoxib (commercially available as Celebrex®), sulindac (commercially available as Clinoril®), oxaprozin (commercially available as Daypro®), piroxicam (commercially available as Feldene®), indomethacin (commercially available as Indocin®), meloxicam (commercially available as Mobic®), mefenamic acid (commercially available as Ponstel®), tolmetin sodium (commercially available as Tolectin®), choline magnesium trisalicylate (commercially available as Trilisate®), diclofenac sodium (commercially available as Voltaren®), and misoprostol (commercially available as Cytotec®). Opiate agonists and antagonists, such as buprenorphine and naloxone are further examples of drugs for use in the present invention.

Other preferred drugs for other preferred active ingredients for use in the present invention include anti-diarrheals such as loperamide (commercially available as Imodium AD®, Imotil®, Kaodene®, Imperim®, Diamode®, QC Anti-Diarrheal®, Health Care America Anti-Diarrheal®, Leader A-D®, and Imogen®), nitazoxanide (commercially available as Alinia®) and diphenoxylate hydrochloride/atropine sulfate (commercially available as Lomotil®), anti-histamines, anti-tussives, decongestants, vitamins, and breath fresheners. Common drugs used alone or in combination for colds, pain, fever, cough, congestion, runny nose and allergies, such as acetaminophen, ibuprofen, chlorpheniramine maleate, dextromethorphan, dextromethorphan HBr, phenylephrine HCl, pseudoephedrine HCl, diphenhydramine and combinations thereof, such as dextromethophan HBr and phenylephrine HCl (available as Triaminic®) may be included in the film compositions of the present invention.

Other active agents useful in the present invention include, but are not limited to alcohol dependence treatment, such as acamprosate calcium (commercially available as Campral®); Allergy treatment medications, such as promethazine hydrochloride (commercially available as Phenergan®), hydrocodone polistirex/chlorpheniramine polistirex (commercially available as Tussionex®), cetirizine hydrochloride (commercially available as Zyrtec®), cetirizine hydrochloride/pseudoephedrine hydrochloride (commercially available as Zyrtec-D®), promethazine hydrochloride/codeine phosphate (commercially available as Phenergan® with Codeine), pemirolast (commercially available as Alamast®), fexofenadine hydrochloride (commercially available as Allegra®), meclizine hydrochloride (commercially available as Antivert®), azelastine hydrochloride (commercially available as Astelin®), nizatidine (commercially available as Axid®), desloratadine (commercially available as Clarinex®), cromolyn sodium (commercially available as Crolom®), epinastine hydrochloride (commercially available as Elestat®), azelastine hydrochloride (commercially available as Optivar®), prednisolone sodium phosphate (commercially available as Orapred ODT®), olopatadine hydrochloride (commercially available as Patanol®), ketotifen fumarate (commercially available as Zaditor®), and montelukast sodium (commercially available as Singulair®); and anti-histamines such as diphenhydramine HCl (available as Benadryl®), loratadine (available as Claritin®), astemizole (available as Hismanal®), nabumetone (available as Relafen®), diphenydramine HCL (available as TheraFlu®) and clemastine (available as Tavist®).

Films of the present invention may further include Alzheimer's treatment medications, such as tacrine hydrochloride (commercially available as Cognex®), galantamine (commercially available as Razadyne®), donepezil hydrochloride (commercially available as Aricept®), rivastigmine tartrate (commercially available as Exelon®), and memantine (commercially available as Namenda®); anemia medication, such as cyanocobalamin (commercially available as Nascobal®); anesthetics, such as antipyrine with benzocaine (commercially available as Auralgan®, Aurodex® and Auroto®); angina medication, such as amlodipine besylate (commercially available as Norvasc®), nitroglycerin (commercially available as Nitro-Bid®, Nitro-Dur®, Nitrolingual®, Nitrostat®, Transderm-Nitro®), isosorbide mononitrate (commercially available as Imdur®), and isosorbide dinitrate (commercially available as Isordil®); anti-tussives such as guaifensin; anti-Alzheimer's agents, such as nicergoline; and Ca^{H}-antagonists such as nifedipine (commercially available as Procardia® and Adalat®).

Actives useful in the present invention may also include anti-asthmatics, such as albuterol sulfate (commercially available as Proventil®), ipratropium bromide (commercially available as Atrovent®), salmeterol xinafoate (commercially available as Serevent®), zafirlukast (commercially available as Accolate®), flunisolide (commercially available as AeroBid®), metaproterenol sulfate (commercially available as Alupent®), albuterol inhalation (commercially available as Ventolin®), terbutaline sulfate (commercially available as Brethine®), formoterol (commercially available as Foradil®), cromolyn sodium (commercially available as Intal®), levalbuterol hydrochloride (commercially available as Xopenex®), zileuton (commercially available as Zyflo®), fluticasone propionate/salmeterol (commercially available as Advair®), albuterol sulfate/triamcinolone acetonide (commercially available as Azmacort®), dimethylxanthine (commercially available as Theophylline®), and beclomethasone (commercially available as Beclovent®, Beconase®, Qvar®, Vancenase®, Vanceril®); and antibacterial medications, such as trimethoprim/sulfamethoxazole (commercially available as Bactrim®), mupirocin (commercially available as Bactroban®), metronidazole (commercially available as Flagyl®), sulfisoxazole acetyl (commercially available as Gantrisin®), bismuth subsalicylate and metronidazole/tetracycline hydrochloride (commercially available as Helidac Therapy®), nitrofurantoin (commercially available as Macrodantin®), norfloxacin (commercially available as Noroxin®), erythromycin ethylsuccinate/Sulfisoxazole acetyl (commercially available as Pediazole®), and levofloxacin (commercially available as Levaquin®).

The present invention may further include one or more Antibiotics, including amoxicillin (commercially available as Amoxil®), ampicillin (commercially available as Omnipen®, Polycillin® and Principen®), amoxicillin/clavulanate potassium (commercially available as Augmentin®), moxifloxacin hydrochloride (commercially available as Avelox®), clarithromycin (commercially available as Biaxin®), ceftibuten (commercially available as Cedax®), cefuroxime axetil (commercially available as Ceftin®), cefprozil (commercially available as Cefzil®), ciprofloxacin hydrochloride (commercially available as Ciloxan® and Cipro®), clindamycin phosphate (commercially available as Cleocin T®), doxycycline hyclate (commercially available as Doryx®), dirithromycin (commercially available as Dynabac®), erythromycin (commercially available as E.E.S.®, E-Mycin®, Eryc®, Ery-Tab®, Erythrocin®, and PCE®), erythromycin topical (commercially available as A/T/S®, Erycette®, T-Stat®), gemifloxacin (commercially available as Factive®), ofloxacin (commercially known as Ocuflox®, Floxin®), telithromycin (commercially available as Ketek®), lomefloxacin hydrochloride (commercially available as Maxaquin®), minocycline hydrochloride (commercially available as Minocin®), fosfomycin tromethamine (commercially available as Monurol®), penicillin with potassium (commercially available as Penicillin VK®, Veetids®), trimethoprim (commercially available as Primsol®), ciprofloxacin hydrochloride (commercially available as Proquin XR®), rifampin, isoniazid and pyrazinamide (commercially available as Rifater®), cefditoren (commercially available as Spectracef®), cefixime (commercially available as Suprax®), tetracycline (commercially available as Achromycin V® and Sumycin®), tobramycin (commercially available as Tobrex®), rifaximin (commercially available as Xifaxan®), azithromycin (commercially available as Zithromax®), azithromycin suspension (commercially available as Zmax®), linezolid (commercially available as Zyvox®), benzoyl peroxide and clindamycin (commercially available as BenzaClin®), erythromycin and benzoyl peroxide (commercially available as Benzamycin®), ciprofloxacin and dexamethasone (commercially available as Ciprodex®), polymyxin B sulfate/neomycin sulfate/hydrocortisone (commercially available as Cortisporin®), colistin sulfate/neomycin sulfate/hydrocortisone acetate/thonzonium bromide (commercially available as Cortisporin-TC Otic®), cephalexin hydrochloride (commercially available as Keflex®), cefdinir (commercially available as Omnicef®), and gatifloxacin (commercially available as Zymar®).

Other useful actives include cancer treatment medications, including cyclophosphamide (commercially available as Cytoxan®), methotrexate (commercially available as Rheumatrex® and Trexal®), tamoxifen citrate (commercially available as Nolvadex®), and anastrozole (commercially available as Arimidex®); anti-coagulants, such as aspirin with extended-release dipyridamole (commercially available as Aggrenox®), warfarin sodium (commercially available as Coumadin®), dipyridamole (commercially available as Persantine®), dalteparin (commercially available as Fragmin®), danaparoid (commercially available as Orgaran®), enoxaparin (commercially available as Lovenox®), heparin (commercially available as Hep-Lock, Hep-Pak, Hep-Pak CVC, Heparin Lock Flush), tinzaparin (commercially available as Innohep®), and clopidogrel bisulfate (commercially available as Plavix®); antiemetics, such as granisetron hydrochloride (commercially available as Kytril®) and nabilone (commercially available as Cesamet®), trimethobenzamide hydrochloride (commercially available as Tigan®), and ondansetron hydrochloride (commercially available as Zofran®); anti-fungal treatment, such as ketoconazole (commercially available as Nizoral®), posaconazole (commercially available as Noxafil®), ciclopirox (commercially available as Penlac®), griseofulvin (commercially available as Gris-PEG®), oxiconazole nitrate (commercially available as Oxistat®), fluconazole (commercially available as Diflucan®), sertaconazole nitrate (commercially available as Ertaczo®), terbinafine hydrochloride (commercially available as Lamisil®), ciclopirox (commercially available as Loprox®), nystatin/triamcinolone acetonide (commercially available as Mycolog-II®), econazole nitrate (commercially available as Spectazole®), itraconazole (commercially available as Sporanox®), and terconazole (commercially available as Terazol®).

Active agents may further include anti-inflammatory medications, such as hydroxychloroquine sulfate (commercially available as Plaquenil®), fluticasone propionate (commercially available as Cutivate®), amcinonide (commercially available as Cyclocort®), methylprednisolone (commercially available as Medrol®), budesonide (commercially available as Entocort EC®), anakinra (commercially available as Kineret®), diflorasone diacetate (commercially available as Psorcon®), and etanercept (commercially available as Enbrel®); antispasmodic medication, such as phenobarbital/hyoscyamine sulfate/atropine sulfate/scopolamine hydrobromide (commercially available as Donnatal®); antiviral treatment, such as oseltamivir phosphate (commercially available as Tamiflu®); anti-parasites medication, including tinidazole (commercially available as Tindamax®); appetite treatment mediations, such as megestrol acetate (commercially available as Megace ES®), phentermine hydrochloride (commercially available as Adipex-P®), and diethylpropion hydrochloride (commercially available as Tenuate®); arthritis medications, including leflunomide (commercially available as Arava®); bladder control medication, such as trospium chloride (commercially available as Sanctura®), desmopressin acetate (commercially available as DDAVP®), tolterodine tartrate (commercially available as Detrol®), oxybutynin chloride (commercially available as Ditropan®), darifenacin (commercially available as Enablex®), and solifenacin succinate (commercially available as VESIcare®); blood vessel constrictors, such as methylergonovine maleate (commercially available as Methergine®); cholesterol lowering medication, including paricalcitol (commercially available as Altocor®), lovastatin, niacin (commercially available as Advicor®), colestipol hydrochloride (commercially available as Colestid®), rosuvastatin calcium (commercially available as Crestor®), fluvastatin sodium (commercially available as Lescol®), atorvastatin calcium (commercially available as Lipitor®), lovastatin (commercially available as Mevacor®), niacin (commercially available as Niaspan®), pravastatin sodium (commercially available as Pravachol®), pavastatin sodium with buffered aspirin (commercially available as Pravigard PAC®), cholestyramine (commercially available as Questran®), simvastatin and niacin (commercially available as Simcor®), atenolol, chlorthalidone (commercially available as Tenoretic®), atenolol (commercially available as Tenormin®), fenofibrate (commercially available as Tricor®), fenofibrate (commercially available as Triglide®), ezetimibe/simvastatin (commercially available as Vytorin®), colesevelam (commercially available as WelChol®), bisoprolol fumarate (commercially available as Zebeta®), ezetimibe (commercially available as Zetia®), bisoprolol fumarate/hydrochlorothiazide (commercially available as Ziac®), and simvastatin (commercially available as Zocor®).

The actives included herein may also include chronic kidney disease medication, such as paricalcitol (commercially available as Zemplar®); contraceptive agents, including etonogestrel (commercially available as Implanon®), norethindrone acetate, ethinyl estradiol (commercially available as Loestrin 24 FE®), ethinyl estradiol, norelgestromin (commercially available as Ortho Evra®), levonorgestrel (commercially available as Plan B®), levonorgestrel and ethinyl estradiol (commercially available as Preven®), levonorgestrel, ethinyl estradiol (commercially available as Seasonique®), and medroxyprogesterone acetate (commercially available as Depo-Provera®); COPD medication, such as arformoterol tartrate (commercially available as Brovana®) and ipratropium bromide, albuterol sulfate (commercially available as Combivent®); cough suppressants, including benzonatate (commercially available as Tessalon®), guaifenesin, codeine phosphate (commercially available as Tussi-Organidin NR®), and acetaminophen, codeine phosphate (commercially available as Tylenol with Codeine®); medication for the treatment of diabetes, including pioglitazone hydrochloride, metformin hydrochloride (commercially available as ACTOplus met®), pioglitazone hydrochloride (commercially available as Actos®), glimepiride (commercially available as Amaryl®), rosiglitazone maleate, metformin hydrochloride (commercially available as Avandamet®), rosiglitazone maleate (commercially available as Avandaryl®), rosiglitazone maleate (commercially available as Avandia®), exenatide (commercially available as Byetta®), chlorpropamide (commercially available as Diabinese®), pioglitazone hydrochloride, glimepiride (commercially available as Duetact®), metformin hydrochloride (commercially available as Glucophage®), glipizide (commercially available as Glucotrol®), glyburide, metformin (commercially available as Glucovance®), metformin hydrochloride (commercially available as Glumetza®), sitagliptin (commercially available as Januvia®), detemir (commercially available as Levemir®), glipizide, metformin hydrochloride (commercially available as Metaglip®), glyburide (commercially available as Micronase®), repaglinide (commercially available as Prandin®), acarbose (commercially available as Precose®), nateglinide (commercially available as Starlix®), pramlintide acetate (commercially available as Symlin®), and tolazamide (commercially available as Tolinase®).

Other useful agents of the present invention may include digestive agents, such as sulfasalazine (commercially available as Azulfidine®), rabeprazole sodium (commercially available as AcipHex®), lubiprostone (commercially available as Amitiza®), dicyclomine hydrochloride (commercially available as Bentyl®), sucralfate (commercially available as Carafate®), lactulose (commercially available as Chronulac®), docusate (commercially available as Colace®), balsalazide disodium (commercially available as Colazal®), losartan potassium (commercially available as Cozaar®), olsalazine sodium (commercially available as Dipentum®), chlordiazepoxide hydrochloride, clidinium bromide (commercially available as Librax®), esomeprazole magnesium (commercially available as Nexium®), famotidine (commercially available as Pepcid®), lansoprazole (commercially available as Prevacid®), lansoprazole and naproxen (commercially available as Prevacid NapraPAC®), amoxicillin/clarithromycin/lansoprazole (commercially available as Prevpac®), omeprazole (commercially available as Prilosec®), pantoprazole sodium (commercially available as Protonix®), metoclopramide hydrochloride (commercially available as Reglan®), cimetidine (commercially available as Tagamet®), ranitidine hydrochloride (commercially available as Zantac®), and omeprazole, sodium bicarbonate (commercially available as Zegerid®); diuretics, including spironolactone, hydrochlorothiazide (commercially available as Aldactazide®), spironolactone (commercially available as Aldactone®). bumetanide (commercially available as Bumex®), torsemide (commercially available as Demadex®), chlorothiazide (commercially available as Diuril®), furosemide (commercially available as Lasix®), metolazone (commercially available as Zaroxolyn®), and hydrochlorothiazide, triamterene (commercially available as Dyazide®).

Agents useful herein may also include treatment for emphysema, such as tiotropium bromide (commercially available as Spiriva®); enema treatments, including aminosalicylic acid (commercially available as Mesalamine® and Rowasa®); epilepsy medications, including valproic acid (commercially available as Depakene®), felbamate (commercially available as Felbatol®), lamotrigine (commercially available as Lamictal®), primidone (commercially available as Mysoline®), oxcarbazepine (commercially available as Trileptal®), zonisamide(commercially available as Zonegran®), levetiracetam (commercially available as Keppra®), and phenytoin sodium (commercially available as Dilantin®).

Erectile dysfunction therapies useful herein include, but are not limited to, drugs for facilitating blood flow to the penis, and for effecting autonomic nervous activities, such as increasing parasympathetic (cholinergic) and decreasing sympathetic (adrenersic) activities. Useful agents for treatment of erectile dysfunction include, for example, those agents available as alprostadil (commercially available as Caverject®), tadalafil (commercially available as Cialis®), vardenafil (commercially available as Levitra®), apomorphine (commercially available as Uprima®), yohimbine hydrochloride (commercially available as Aphrodyne®, Yocon®), and sildenafil citrate (commercially available as Viagra®).

Agents useful herein may further include eye medications and treatment, such as dipivefrin hydrochloride (commercially available as Propine®), valganciclovir (commercially available as Valcyte®), bromfenac (commercially available as Xibrom®), fluorometholone (commercially available as FML®), pilocarpine hydrochloride (commercially available as Pilocar®), cyclosporine (commercially available as Restasis®), brimonidine tartrate (commercially available as Alphagan P®), dorzolamide hydrochloride/timolol maleate (commercially available as Cosopt®), bimatoprost (commercially available as Lumigan®), timolol maleate (available as Timoptic®), travoprost (commercially available as Travatan®), latanoprost (commercially available as Xalatan®), echothiophate iodide (commercially available as Phospholine Iodide®), and ranibizumab (commercially available as Lucentis®); fluid controllers, such as acetazolamide (commercially available as Diamox®); gallstone medications, including ursodiol (commercially available as Actigall®); medication for the treatment of gingivitis, including chlorhexidine gluconate (commercially available as Peridex®); headache medications, including butalbital/codeine phosphate/aspirin/caffeine (commercially available as Fiornal® with Codeine), naratriptan hydrochloride (commercially available as Amerge®), almotriptan (commercially available as Axert®), ergotamine tartrate/caffeine (commercially available as Cafergot®), butalbital/acetaminophen/caffeine (commercially available as Fioricet®), butalbital/aspirin/caffeine (commercially available as Fiorinal®), frovatriptan succinate (commercially available as Frova®), rizatriptan benzoate (commercially available as Maxalt®), isometheptene mucate/dichloralphenazone/acetaminophen (commercially available as Midrin®), dihydroergotamine mesylate (commercially available as Migranal®), eletriptan hydrobromide (commercially available as Relpax®), and zolmitriptan (commercially available as Zomig®); and heart treatments, including quinidine sulfate, isosorbide dinitrate/hydralazine hydrochloride (commercially available as BiDil®), digoxin (commercially available as Lanoxin®), flecainide acetate (commercially available as Tambocor®), mexiletine hydrochloride (commercially available as Mexitil®), disopyramide phosphate (commercially available as Norpace®), procainamide hydrochloride (commercially available as Procanbid®), and propafenone (commercially available as Rythmol®).

Other useful agents include hepatitis treatments, including entecavir (commercially available as Baraclude®), hepatitis B immune globulin (commercially available as HepaGam B®), and copegus/rebetol/ribasphere/vilona/virazole (commercially available as Ribavirin®); herpes treatments, including valacyclovir hydrochloride (commercially available as Valtrex®), penciclovir (commercially available as Denavir®), acyclovir (commercially available as Zovirax®), and famciclovir (commercially available as Famvir®); treatment for high blood pressure, including enalaprilat (available as Vasotec®), captopril (available as Capoten®) and lisinopril (available as Zestril®), verapamil hydrochloride (available as Calan®), ramipril (commercially available as Altace®), olmesartan medoxomil (commercially available as Benicar®), amlodipine/atorvastatin (commercially available as Caduet®), nicardipine hydrochloride (commercially available as Cardene®), diltiazem hydrochloride (commercially available as Cardizem®), quinapril hydrochloride (commercially available as Accupril®), quinapril hydrochloride/hydrochlorothiazide (commercially available as Accuretic®), perindopril erbumine (commercially available as Aceon®), candesartan cilexetil (commercially available as Atacand®), candesartan cilexetil/hydrochlorothiazide (commercially available as Atacand HCT®), irbesartan/hydrochlorothiazide (commercially available as Avalide®), irbesartan (commercially available as Avapro®), amlodipine besylate/olmesartan medoxomil (commercially available as Azor®), levobunolol hydrochloride (commercially available as Betagan®), betaxolol hydrochloride (commercially available as Betoptic®), nebivolol (commercially available as Bystolic®), captopril/hydrochlorothiazide (commercially available as Capozide®), doxazosin mesylate (commercially available as Cardura®), clonidine hydrochloride (commercially available as Catapres®), carvedilol (commercially available as Coreg®), nadolol (commercially available as Corgard®), nadolol/bendroflumethiazide (commercially available as Corzide®), valsartan (commercially available as Diovan®), isradipine (commercially available as DynaCirc®), wytensin. (commercially available as Guanabenz acetate®), tenex (commercially available as Guanfacine hydrochloride®), losartan potassium/hydrochlorothiazide (commercially available as Hyzaar®), propranolol hydrochloride (commercially available as Indera®), propranolol hydrochloride/hydrochlorothiazide (commercially available as Inderide®), eplerenone (commercially available as Inspra®), ambrisentan (commercially available as Letairis®), enalapril maleate/felodipine (commercially available as Lexxel®), metoprolol tartrate (commercially available as Lopressor®), benazepril hydrochloride (commercially available as Lotensin®), benazepril hydrochloride/hydrochlorothiazide (commercially available as Lotensin HCT®), amlodipine/benazepril hydrochloride (commercially available as Lotrel®), indapamide (commercially available as Lozol®), trandolapril (commercially available as Mavik®), telmisartan (commercially available as Micardis®), telmisartan/hydrochlorothiazide (commercially available as Micardis HCT®), prazosin hydrochloride (commercially available as Minipress®), amiloride, hydrochlorothiazide (commercially available as Moduretic®), fosinopril sodium (commercially available as ZZXT Monopril®), fosinopril sodium/hydrochlorothiazide (commercially available as Monopril-HCT®), pindolol (commercially available as Visken®), felodipine (commercially available as Plendil®), sildenafil citrate (commercially available as Revatio®), Nisoldipine (commercially available as Sular®), trandolapril/verapamil hydrochloride (commercially available as Tarka®), aliskiren (commercially available as Tekturna®), eprosartan mesylate (commercially available as Teveten®), eprosartan mesylate/hydrochlorothiazide (commercially available as Teveten HCT®), moexipril hydrochloride/hydrochlorothiazide (commercially available as Uniretic®), moexipril hydrochloride (commercially available as Univasc®), enalapril maleate/hydrochlorothiazide (commercially available as Vaseretic®), and lisinopril/hydrochlorothiazide (commercially available as Zestoretic®).

The present invention may include agents useful in the medication for the treatment of HIV/AIDS, such as amprenavir (commercially available as Agenerase®), tipranavir (commercially available as Aptivus®), efavirenz/emtricitabine/tenofovir (commercially available as Atripla®), lamivudine/zidovudine (commercially available as Combivir®), indinavir sulfate (commercially available as Crixivan®), lamivudine (commercially available as Epivir®), saquinavir (commercially available as Fortovase®), zalcitabine (commercially available as Hivid®), lopinavir/ritonavir (commercially available as Kaletra®), fosamprenavir calcium (commercially available as Lexiva®), ritonavir (commercially available as Norvir®), zidovudine (commercially available as Retrovir®), atazanavir sulfate (commercially available as Reyataz®), efavirenz (commercially available as Sustiva®), abacavir/lamivudine/zidovudine (commercially available as Trizivir®), didanosine (commercially available as Videx®), nelfinavir mesylate (commercially available as Viracept®), nevirapine (commercially available as Viramune®), tenofovir disoproxil fumarate (commercially available as Viread®), stavudine (commercially available as Zerit®), and abacavir sulfate (commercially available as Ziagen®); homocysteiene removers, including betaine anhydrous (commercially available as Cystadane®); medications, such as insulin (commercially available as Apidra®, Humalog®, Humulin®, Iletin®, and Novolin®); and HPV treatment, such as Human papillomavirus vaccine (commercially available as Gardasil®); immunosuppressants, including cyclosporine (commercially available as Gengraf®, Neoral®, Sandimmune®, and Apo-Cyclosporine®).

Agents useful in the present invention may further include prolactin inhibitors, such as bromocriptine mesylate (commercially available as Parlodel®); medications for aiding in stress tests, such as regadenoson (commercially available as Lexiscan®); baldness medication, including finasteride (commercially available as Propecia® and Proscar®); pancreatitis treatment, such as gemfibrozil (commercially available as Lopid®); hormone medications, such as norethindrone acetate/ethinyl estradiol (commercially available as femHRT®), goserelin acetate (commercially available as Zoladex®), progesterone gel (commercially available as Prochieve®), progesterone (commercially available as Prometrium®), calcitonin-salmon (commercially available as Miacalcin®), calcitriol (commercially available as Rocaltrol®), synthroid (commercially available as Levothroid®, Levoxyl®, Unithroid®), testosterone (commercially available as Testopel®, Androderm®, Testoderm®, and AndroGel®); menopause medication, such as estradiol/norethindrone acetate (commercially available as Activella®), drospirenone/estradiol (commercially available as Angeliq®), estradiol/levonorgestrel (commercially available as Climara Pro®), estradiol/norethindrone acetate (commercially available as CombiPatch®), estradiol (commercially available as Estrasorb®, Vagifem® and EstroGel®), esterified estrogens and methyltestosterone (commercially available as Estratest®), estrogen (commercially available as Alora®, Climara®, Esclim®, Estraderm®, Vivelle®, Vivelle-Dot®), estropipate (commercially available as Ogen®), conjugated estrogens (commercially available as Premarin®), and medroxyprogesterone acetate (commercially available as Provera®); menstrual medications, including leuprolide acetate (commercially available as Lupron Depot), and norethindrone acetate (commercially available as Aygestin); and muscle relaxants, including cyclobenzaprine hydrochloride (commercially available as Flexeril®), tizanidine (commercially available as Zanaflex®), and hyoscyamine sulfate (commercially available as Levsin®).

Agents useful herein may also include osteoporosis medications, including ibrandronate sodium (commercially available as Boniva®), risedronate (commercially available as Actonel®), raloxifene hydrochloride (commercially available as Evista®, Fortical®), and alendronate sodium (commercially available as Fosamax®); ovulation enhancers, including clomiphene citrate (commercially available as Serophene®, Clomid®, Serophene®); Paget's disease treatment, such as etidronate disodium (commercially available as Didronel®); pancreatic enzyme deficiency medications, such as pancrelipase (commercially available as Pancrease®); medication for the treatment of Parkinson's disease, such as pramipexole dihydrochloride (commercially available as Mirapex®), ropinirole hydrochloride (commercially available as Requip®), carbidopa/levodopa (commercially available as Sinemet CR®), carbidopa/levodopa/entacapone (commercially available as Stalevo®), selegiline hydrochloride (commercially available as Zelapar®), rasagiline (commercially available as Azilect®), entacapone (commercially available as Comtan®), and selegiline hydrochloride (commercially available as Eldepryl®); prostate medication, including flutamide (commercially available as Eulexin®), nilutamide (commercially available as Nilandron®), dutasteride (commercially available as Avodart®), tamsulosin hydrochloride (commercially available as Flomax®), terazosin hydrochloride (commercially available as Hytrin®), and alfuzosin hydrochloride (commercially available as UroXatral®).

Films of the present invention may further include psychiatric medications, including alprazolam (available as Niravam®, Xanax®), clozopin (available as Clozaril®), haloperidol (available as Haldol®), fluoxetine hydrochloride (available as Prozac®), sertraline hydrochloride (available as Zoloft®), and paroxtine hydrochloride (available as Paxil®), aripiprazole (commercially aavialbe as Abilify®), Amphetamines and methamphetamines (commercially available as Adderall® and Desoxyn®), clomipramine hydrochloride (commercially available as Anafranil®), Buspirone hydrochloride (commercially available as BuSpar®), citalopram hydrobromide (commercially available as Celexa®), duloxetine hydrochloride (commercially available as Cymbalta®), methylphenidate (commercially available as Ritalin, Daytrana®), divalproex sodium (Valproic acid) (commercially available as Depakote®), dextroamphetamine sulfate (commercially available as Dexedrine®), venlafaxine hydrochloride (commercially available as Effexor®), selegiline (commercially available as Emsam®), carbamazepine (commercially available as Equetro®), lithium carbonate (commercially available as Eskalith®), fluvoxamine maleate/dexmethylphenidate hydrochloride (commercially available as Focalin®), ziprasidone hydrochloride (commercially available as Geodon®), ergoloid mesylates (commercially available as Hydergine®), escitalopram oxalate (commercially available as Lexapro®), chlordiazepoxide (commercially available as Librium®), molindone hydrochloride (commercially available as Moban®), phenelzine sulfate (commercially available as Nardil®), thiothixene (commercially available as Navane®), desipramine hydrochloride (commercially available as Norpramin®), benzodiazepines (such as those available as Oxazepam®), nortriptyline hydrochloride (commercially available as Pamelor®), tranylcypromine sulfate (commercially available as Parnate®), prochlorperazine, mirtazapine (commercially available as Remeron®), risperidone (commercially available as Risperdal®), quetiapine fumarate (commercially available as Seroquel®), doxepin hydrochloride (commercially available as Sinequan®), atomoxetine hydrochloride (commercially available as Strattera®), trimipramine maleate (commercially available as Surmontil®), olanzapine/fluoxetine hydrochloride (commercially available as Symbyax®), imipramine hydrochloride (commercially available as Tofranil®), protriptyline hydrochloride (commercially available as Vivactil®), bupropion hydrochloride (commercially available as Wellbutrin®, Wellbutrin SR®, and Wellbutrin XR®), and olanzapine (commercially available as Zyprexa®).

Agents useful herein may also include uric acid reduction treatment, including allopurinol (commercially available as Zyloprim®); seizure medications, including gabapentin (commercially available as Neurontin®), ethotoin (commercially available as Peganone®), and topiramate (commercially available as Topamax®); treatment for shingles, such as zoster vaccine live (commercially available as Zostavax®); skin care medications, including calcipotriene (commercially available as Dovonex®), isotretinoin (commercially available as Accutane®), hydrocortisone/iodoquinol (commercially available as Alcortin®), sulfacetamide sodium/sulfur (commercially available as Avar®), azelaic acid (commercially available as Azelex®, Finacea®), benzoyl peroxide (commercially available as Desquam-E®), adapalene (commercially available as Differing), fluorouracil (commercially available as Efudex®), pimecrolimus (commercially available as Elidel®), topical erythromycin (commercially available as A/T/S®, Erycette®, T-Stat®), hydrocortisone (commercially available as Cetacort®, Hytone®, Nutracort®), metronidazole (commercially available as MetroGel®), doxycycline (commercially available as Oracea®), tretinoin (commercially available as Retin-A® and Renova®), mequinol/tretinoin (commercially available as Solagé®), acitretin (commercially available as Soriatane®), calcipotriene hydrate/betamethasone dipropionate (commercially available as Taclonex®), tazarotene (commercially available as Tazorac®), fluocinonide (commercially available as Vanos®), desonide (commercially available as Verdeso®), miconazole nitrate/Zinc oxide (commercially available as Vusion®), ketoconazole (commercially available as Xolegel®), and efalizumab (commercially available as Raptiva®).

Other agents useful herein may include Sleep disorder medications, including zaleplon (available as Sonata®) and eszopiclone (available as Lunesta®), zolpidem tartrate (commercially available as Ambien®, Ambien CR®), lorazepam (commercially available as Ativan®), flurazepam hydrochloride (commercially available as Dalmane®), triazolam (commercially available as Halcion®), clonazepam (commercially available as Klonopin®), barbituates, such as Phenobarbital®), Modafinil (commercially available as Provigil®), temazepam (commercially available as Restoril®), ramelteon (commercially available as Rozerem®), clorazepate dipotassium (commercially available as Tranxene®), diazepam (commercially available as Valium®), quazepam (commercially available as Doral®), and estazolam (commercially available as ProSom®); smoking cessation medications, such as varenicline (commercially available as Chantix®), nicotine, such as Nicotrol®, and bupropion hydrochloride (commercially available as Zyban®); and steroids, including alclometasone dipropionate (commercially available as Aclovate®), betamethasone dipropionate (commercially available as Diprolene®), mometasone furoate (commercially available as Elocon®), fluticasone (commercially available as Flonase®, Flovent®, Flovent Diskus®, Flovent Rotadisk®), fluocinonide (commercially available as Lidex®), mometasone furoate monohydrate (commercially available as Nasonex®), desoximetasone (commercially available as Topicort®), clotrimazole/betamethasone dipropionate (commercially available as Lotrisone®), prednisolone acetate (commercially available as Pred Forte®, Prednisone®, Budesonide Pulmicort®, Rhinocort Aqua®), prednisolone sodium phosphate (commercially available as Pediapred®), desonide (commercially available as Tridesilon®), and halobetasol propionate (commercially available as Ultravate®).

Films of the present invention may further include agents useful for thyroid disease treatment, such as hormones TC and TD (commercially available as Armour Thyroid®); potassium deficiency treatment, including potassium chloride (commercially available as Micro-K®); triglycerides regulators, including omega-3-acid ethyl esters (commercially available as Omacor®); urinary medication, such as phenazopyridine hydrochloride (commercially available as Pyridium®) and methenamine, methylene blue/phenyl salicylate/benzoic acid/atropine sulfate/hyoscyamine (commercially available as Urised®); prenatal vitamins (commercially available as Advanced Natalcare®, Materna®, Natalins®, Prenate Advance®); weight control medication, including orlistat (commercially available as Xenical®) and sibutramine hydrochloride (commercially available as Meridia®).

The popular H₂-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

Botanicals, herbals and minerals also may be added to the film. Examples of botanicals include, without limitation: roots; barks; leaves; stems; flowers; fruits; tobacco; sunflower seeds; snuff; and combinations thereof.

An anti-oxidant may also be added to the film to prevent the degradation of an active, especially where the active is photosensitive.

The bioactive active substances employed in the present invention may include beneficial bacteria. More specifically, certain bacteria normally exist on the surface of the tongue and in the back of the throat. Such bacteria assist in the digestion of food by breaking down proteins found in the food. It may be desirable, therefore, to incorporate these bacteria into the oral film products of the present invention.

It also may be desirable to include actives for treating breath malodor and related oral care conditions, such as actives which are effective in suppressing microorganisms. Because breath malodor can be caused by the presence of anaerobic bacteria in the oral cavity, which generate volatile sulfur compounds, components that suppress such microorganisms may be desirable. Examples of such components include antimicrobials such as triclosan, chlorine dioxide, chlorates, and chlorites, among others. The use of chlorites, particularly sodium chlorite, in oral care compositions such as mouth rinses and toothpastes is taught in U.S. Pat. Nos. 6,251,372, 6,132,702, 6,077,502, and U.S. Publication No. 2003/0129144, all of which are incorporated herein by reference. Such components are incorporated in amounts effective to treat malodor and related oral conditions.

Cosmetic active agents may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

Also color additives can be used in preparing the films. Such color additives include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium. These oxides may be added in concentrations ranging from about 0.001 to about 10%, and, desirably from about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

The films containing flavorings may be added to provide a hot or cold flavored drink or soup. These flavorings include, without limitation, tea and soup flavorings such as beef and chicken.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

When the active is combined with the polymer in the solvent, the type of matrix that is formed depends on the solubility's of the active and the polymer. If the active and/or polymer are soluble in the selected solvent, this may form a solution. However, if the components are not soluble, the matrix may be classified as an emulsion, a colloid, or a suspension.

### Dosages

The film products of the present invention are capable of accommodating a wide range of amounts of the active ingredient. The films are capable of providing an accurate dosage amount (determined by the size of the film and concentration of the active in the original polymer/water combination) regardless of whether the required dosage is high or extremely low. Therefore, depending on the type of active or pharmaceutical composition that is incorporated into the film, the active amount may be as high as about 300 mg, desirably up to about 150 mg or as low as the microgram range, or any amount therebetween.

The film products and methods of the present invention are well suited for high potency, low dosage drugs. This is accomplished through the high degree of uniformity of the films. Therefore, low dosage drugs, particularly more potent racemic mixtures of actives are desirable.

### Optional Components

A variety of other components and fillers may also be added to the films of the present invention. These may include, without limitation, the following: surfactants; plasticizers which assist in compatibilizing the components within the mixture; polyalcohols; anti-foaming agents, such as silicone-containing compounds, which promote a smoother film surface by releasing oxygen from the film; and thermo-setting gels such as pectin, carageenan, and gelatin, which help in maintaining the dispersion of components, and combinations thereof.

The variety of additives that can be incorporated into the inventive compositions may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to about 50% and more desirably within the range of 3% to about 20% based on the weight of all components.

Further additives may be inorganic fillers, such as the oxides of magnesium aluminum, silicon, titanium and the like, desirably in a concentration range of about 0.02% to about 3% by weight and, desirably, about 0.02% to about 1% based on the weight of all components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e., with C₁₂-, C₁₄-, C₁₆-, C₁₈-, C₂₀- and C₂₂-fatty acids.

The total amounts used of the fats, mono-, di-glycerides and/or lecithins are up to about 5% and preferably within the range of from about 0.5% to about 2% by weight of the total composition.

It is further useful to add silicon dioxide, calcium silicate, or titanium dioxide in a concentration of about 0.02% to about 1% by weight of the total composition. These compounds act as texturizing agents.

These additives are to be used in amounts sufficient to achieve their intended purpose. Generally, the combination of certain of these additives will alter the overall release profile of the active ingredient and can be used to modify, i.e., impede or accelerate the release.

Lecithin is one surface active agent for use in the present invention. Lecithin can be included in the feedstock in an amount of from about 0.25% to about 2.00% by weight. Other surface active agents, i.e., surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL, which is commercially available from BASF, are also useful. Carbowax™ is yet another modifier which is very useful in the present invention. Tweens™ or combinations of surface active agents may be used to achieve the desired hydrophilic-lipophilic balance (“HLB”). The present invention, however, does not require the use of a surfactant. Films or film-forming compositions of the present invention may be essentially free of a surfactant while still providing the desirable uniformity features of the present invention.

It may be further useful to add polydextrose to the films of the present invention. Polydextrose serves as a filler and solubility enhancer, i.e., it increases the dissolution time of the films in the oral cavity.

It will be understood that any modifier that enhances the procedure and/or product of the present invention are identified. Applicants intend to include all such additional modifiers within the scope of the invention claimed herein.

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

### Forming the Film

The films of the present invention may be formed into a sheet prior to drying. After the desired components are combined to form a multi-component matrix, including the polymer, water, and an active or other components as desired, the combination is formed into a sheet or film, by any method known in the art such as extrusion, coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer.

Although a variety of different film-forming techniques may be used, it is desirable to select a method that will provide a flexible film, such as reverse roll coating. The flexibility of the film allows for the sheets of film to be rolled and transported for storage or prior to being cut into individual dosage forms. Desirably, the films will also be self-supporting or, in other words, able to maintain their integrity and structure in the absence of a separate support. Furthermore, the films of the present invention may be selected of materials that are edible or ingestible.

Coating or casting methods are particularly useful for the purpose of forming the films of the present invention. Specific examples include reverse roll coating, gravure coating, immersion or dip coating, metering rod or meyer bar coating, slot die or extrusion coating, gap or knife over roll coating, air knife coating, curtain coating, or combinations thereof, especially when a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

Offset Gravure is common, where the coating is deposited on an intermediate roller before transfer to the substrate.

In the simple process of immersion or dip coating, the substrate is dipped into a bath of the coating, which is normally of a low viscosity to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

In the slot die process, the coating is squeezed out by gravity or under pressure through a slot and onto the substrate. If the coating is 100% solids, the process is termed “Extrusion” and in this case, the line speed is frequently much faster than the speed of the extrusion. This enables coatings to be considerably thinner than the width of the slot.

The gap or knife over roll process relies on a coating being applied to the substrate which then passes through a “gap” between a “knife” and a support roller. As the coating and substrate pass through, the excess is scraped off.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face.

Moreover, the films of the present invention may contain particles that are sensitive to temperature, such as flavors, which may be volatile, or drugs, which may have a low degradation temperature. In such cases, the drying temperature may be decreased while increasing the drying time to adequately dry the uniform films of the present invention. Furthermore, bottom drying also tends to result in a lower internal film temperature as compared to top drying. In bottom drying, the evaporating vapors more readily carry heat away from the film as compared to top drying which lowers the internal film temperature. Such lower internal film temperatures often result in decreased drug degradation and decreased loss of certain volatiles, such as flavors.

As described above, the dissolvable film dosage form may contain indicia. Indicia may be useful when incorporated on the dissolvable film dosage form since it can provide information to a consumer or manufacturer regarding the dissolvable film's manufacturer, expiration date, active ingredient and the like.

The features and advantages of the present invention are more fully shown by the following examples, which are provided for purposes of illustration, and are not to be construed as limiting the invention in any way.

### EXAMPLES

### Example 1

Table 1 below is an example of a film composition of the present invention.

TABLE 1

| | Weight (g unless |
|---|---|
| Component | otherwise indicated) |
| Hydroxypropylcellulose | 6.00 |
| Polyethylene oxide | 2.00 |
| Sucralose¹ | 0.84 |
| Magna sweet² | 0.09 |
| Mixture of microcrystalline | 0.18 |
| cellulose and sodium | |
| carboxymethylcellulose³ | |
| Precipitated calcium carbonate | 1.55 |
| Sildenafil⁴ | 2.91 |
| Peppermint & bittermint flavor | 1.75 |
| Prosweet⁵ | 0.44 |
| Masking flavor⁶ | 1.31 |
| N,2,3-trimethyl-2- | 0.075 |
| isopropylbutanamide⁷ | |
| Simethicone⁸ | 0.035 |
| Water | 32.5 |
| Blue food coloring | 3 drops |

¹Available from McNeil Nutritional
²Taste-masking flavor, available from Mafco Worldwide Corp.
³Avicel CL-611, available from FMC Biopolymer
⁴Available from Pfizer, Inc. as Viagra ®
⁵Taste-masking flavor, available from Virginia Dare
⁶Available from Ungerer and Co.
⁷Cooling agent
⁸Available from Sentry

The above ingredients are combined by mixing until a uniform mixture is achieved, and then is cast into two films on release paper using a K-Control Coater with a 350 micron smooth bar. The film is dried. Edible film dosage forms of the present invention are prepared by applying indicia in the form of a barcode on a surface of the film using a rotogravure printing technique.

### Example 2

Another example of a film composition is prepared according to Example 1. In this example, the indicia is printed on the surface of the film using an inkjet printing technique.

### Example 3

Another example of a film composition is prepared according to Example 1. In this example, the indicia is not printed on the surface of the film. After the film is dried, it is cut into individual dosage forms. The individual dosage forms are enclosed in a sealed pouch. The indicia is then applied on a surface of the sealed pouch using a rotogravure printing technique. The resulting product is a packaged edible film dosage form.

### Example 4

Table 2 below is an example of a film composition of the present invention.

TABLE 2

| | Weight (g unless |
|---|---|
| Component | otherwise indicated) |
| Hydroxypropylcellulose | 6.00 |
| Polyethylene oxide | 2.00 |
| Sucralose¹ | 0.84 |
| Magna sweet² | 0.09 |
| Mixture of microcrystalline | 0.18 |
| cellulose and sodium | |
| carboxymethylcellulose³ | |
| Precipitated calcium carbonate | 1.55 |
| Sildenafil⁴ | 2.91 |
| Peppermint & bittermint flavor | 1.75 |
| Prosweet⁵ | 0.44 |
| Masking flavor⁶ | 1.31 |
| N,2,3-trimethyl-2- | 0.075 |
| isopropylbutanamide⁷ | |
| Simethicone⁸ | 0.035 |
| Water | 32.5 |
| Blue food coloring | 3 drops |

¹Available from McNeil Nutritional
²Taste-masking flavor, available from Mafco Worldwide Corp.
³Avicel CL-611, available from FMC Biopolymer
⁴Available from Pfizer, Inc. as Viagra ®
⁵Taste-masking flavor, available from Virginia Dare
⁶Available from Ungerer and Co.
⁷Cooling agent
⁸Available from Sentry

The above ingredients are combined by mixing until a uniform mixture is achieved, and then the mixture is cast into two films on release paper using a K-Control Coater with a 350 micron smooth bar. The film is dried.

Edible film dosage forms of the present invention are prepared by applying the indicia composition as the name of the active ingredient on a surface of the film using an inkjet printing technique.

What is claimed is:

1. An edible film dosage form comprising:
(a) a film composition comprising:
a film-forming polymer; and
(ii) at least one active component;
wherein said film composition has at least one surface; and
(b) indicia associated with said at least one surface of said film composition, wherein said at least one indicia includes information relevant to said film composition.

2. The edible film dosage form of claim 1, wherein said indicia comprises a barcode.

3. The edible film dosage form of claim 2, wherein said barcode when decoded provides information selected from the group consisting of ingredients, identification information, manufacturing information, ingredient information, dosage information, safety information, side effect information, information relevant to studies conducted on the active or dosage and combinations thereof.

4. The edible film dosage form of claim 2, wherein said barcode includes batch number, lot number, manufacturing date, expiration date and product name.

5. The edible film dosage form of claim 2, wherein said barcode comprises a communication which is invisible to the naked eye.

6. The edible film dosage form of claim 2, wherein said barcode comprises a communication which provides a quality control check.

7. The edible film dosage form of claim 1, wherein said at least one indicia is printed on the surface.

8. The edible film dosage form of claim 1, wherein said indicia is representative of the content of one or more active components.

9. The edible film dosage form of claim 11, wherein the indicia is representative of the relative content of two or more active components.

10. The edible film dosage form of claim 1, wherein said indicia is selected from the group consisting of patterns, stripes, straight stripes, wavy stripes, letters, numbers, dots, spots, specks, geometric shapes, complex shapes, and combinations thereof.

11. The edible film dosage form of claim 1, wherein said indicia is speckled.

12. The edible film dosage form of claim 1, wherein said edible film dosage form comprises at least two indicia, wherein a first indicia is selected from the group consisting of a barcode, patterns, stripes, straight stripes, wavy stripes, letters, numbers, dots, spots, specks, geometric shapes, complex shapes, and combinations thereof and a second indicia is selected from the group consisting of barcode patterns, straight stripes, wavy stripes, letters, numbers, dots, spots, specks, geometric shapes, complex shapes, and combinations thereof, wherein said first indicia is the same or different from said second indicia.

13. The edible film dosage form of claim 1, wherein said edible film dosage form comprises two indicia, wherein a first indicia comprises a first coloring agent and a second indicia comprises a second coloring agent, wherein said first coloring agent is the same or different from said second coloring agent.

14. The edible film dosage form of claim 1, wherein said film composition further comprises an excipient.

15. The edible film dosage form of claim 14, wherein said excipient comprises a first coloring agent and said indicia comprises a second coloring agent, wherein said first coloring agent is different from said second coloring agent.

16. The edible film dosage form of claim 14, wherein said excipient comprises a first coloring agent and said indicia comprises a second coloring agent, wherein said first coloring agent contrasts with said second coloring agent.

17. The edible film dosage form of claim 14, wherein said excipient is selected from the group consisting of colors, flavors, sweeteners, fillers, plasticizers, surfactants, polyols, and combinations thereof.

18. The edible film dosage form of claim 14, wherein said at least one indicia represents the presence of multiple active components in said film dosage form.

19. The edible film dosage form of claim 1, wherein said active component is a member selected from the group consisting of medicaments, flavors, fragrances, enzymes, preservatives, sweetening agents, colorants, spices, vitamins, and combinations thereof.

20. The edible film dosage form of claim 1, wherein said at least one indicia is printed on said at least one surface of said film composition.

21. The edible film dosage form of claim 1, wherein said at least one indicia is printed by a rotogravure printing technique.

22. The edible film dosage form of claim 1, wherein said at least one indicia is embedded in said at least one surface of said film composition.

23. The edible film dosage form of claim 1, wherein said at least one indicia is in contact with said at least one surface of said film composition.

24. The edible film dosage form of claim 1, wherein said at least one indicia comprises edible ink.

25. The edible film dosage form of claim 1, wherein said at least one indicia represents the ratio of a combination of active components present in said edible film dosage form.

26. The edible film dosage form of claim 1, wherein said indicia comprises a first coloring agent and said film composition comprises a second coloring agent, wherein said first coloring agent and said second coloring agent are different.

27. The edible film dosage form of claim 1, wherein said indicia covers the entire portion of said at least one surface of said film composition.

28. The edible film dosage form of claim 1, wherein said indicia comprises a first coloring agent and said film composition comprises a second coloring agent, wherein said first coloring agent and said second coloring agent are contrasting.

29. An edible film dosage form comprising:
(a) a film composition comprising:
(i) a film-forming polymer; and
(ii) at least one active component;
wherein said film composition has at least one surface; and
(b) at least one indicia associated with said at least one surface of said film composition, wherein said at least one indicia comprises a monitoring agent.

30. The edible film dosage of claim 29, wherein said monitoring agent is used for quality control.

31. An edible film dosage form comprising:
(a) a film composition comprising:
(i) a film-forming polymer; and
(ii) at least one active component;
wherein said film composition has at least one surface; and
(b) at least one indicia associated with said at least one surface of said film composition, wherein said at least one indicia comprises multiple levels of information.

32. The edible film dosage form of claim 31, wherein said indicia comprises a primary level of information and a secondary level of information, wherein said primary level of information directly comprises a direct meaning and said secondary level of information comprises an indirect meaning.

33. The edible film dosage form of claim 31, wherein said indicia comprises a primary level of information, a secondary level of information, and a tertiary level of information, wherein said primary level of information directly conveys a meaning and said secondary and tertiary level of information must be deciphered.

34. A pharmaceutical film dosage form comprising:
(a) a film composition comprising:
(i) at least one water-soluble polymer; and
(ii) at least one pharmaceutical active,
wherein said film composition has at least one surface; and
(b) at least one indicia associated with at least one surface of said film composition, wherein said at least one indicia provides information.

35. The pharmaceutical film dosage form of claim 34, wherein said information is in the form of a barcode.

36. The pharmaceutical film dosage form of claim 35, wherein said barcode comprises a communication selected from the group consisting of ingredients, lack of ingredients, side effects, a product name, a product manufacturer, a dosage amount, a lot number, a batch number, a date, and combinations thereof.

37. The pharmaceutical film dosage form of claim 35, wherein said barcode comprises a communication which is invisible to the naked eye.

38. The pharmaceutical film dosage form of claim 35, wherein said barcode comprises a communication which provides a quality control check.

39. The pharmaceutical film dosage form of claim 35, wherein said information is a communication selected from the group consisting of a barcode, a product name, a product manufacture, a dosage amount, a lot number, a batch number, a date, and combinations thereof.

40. The pharmaceutical film dosage form of claim 35, wherein said information corresponds to said at least one active component.

41. The pharmaceutical film dosage form of claim 34, wherein the indicia is printed on the surface.

42. The pharmaceutical film dosage form of claim 34, wherein said indicia is selected from the group consisting of patterns, straight stripes, wavy stripes, letters, numbers, dots, spots, specks, geometric shapes, complex shapes, and combinations thereof.

43. The pharmaceutical film dosage form of claim 34, wherein said film composition comprises at least two indicia, wherein a first indicia is selected from the group consisting of patterns, straight stripes, wavy stripes, letters, numbers, dots, spots, specks, geometric shapes, complex shapes, and combinations thereof and a second indicia is selected from the group consisting of patterns, straight stripes, wavy stripes, letters, numbers, dots, spots, specks, geometric shapes, complex shapes, and combinations thereof, wherein said first indicia is the same or different from said second indicia.

44. The pharmaceutical film dosage form of claim 34, wherein said film composition comprises two indicia, wherein a first indicia comprises a first coloring agent and a second indicia comprises a second coloring agent, wherein said first coloring agent is the same or different from said second coloring agent.

45. The pharmaceutical film dosage form of claim 34, wherein said indicia represents the presence of multiple active components in said film dosage form.

46. The pharmaceutical film dosage form of claim 34, wherein said indicia is printed on said at least one surface of said film composition.

47. The pharmaceutical film dosage form of claim 34, wherein said at least one indicia is printed by a rotogravure printing technique.

48. The pharmaceutical film dosage form of claim 34, wherein said indicia is embedded in said at least one surface of said film composition.

49. The pharmaceutical film dosage form of claim 34, wherein said indicia is in contact with said at least one surface of said film composition.

50. The pharmaceutical film dosage form of claim 34, wherein said indicia comprises edible ink.

51. The pharmaceutical film dosage form of claim 34, wherein said at least one indicia represents the ratio of a combination of active components present in said pharmaceutical film dosage form.

52. A packaged edible film dosage form comprising:
(a) at least one film composition comprising:
(i) a film forming polymer; and
(ii) at least one active component, said at least one film composition being enclosed within a sealed pouch, wherein said sealed pouch comprises a top layer, a bottom layer, an inner cavity and has at least one surface; and
(b) at least one indicia associated with said at least one surface of said sealed pouch, wherein said at least one indicia provides at least one level of information.

53. The packaged edible film dosage form of claim 52, wherein said sealed pouch further comprises a transparent portion.

54. The packaged edible film dosage form of claim 52, wherein said indicia is invisible to the naked eye.

55. The packaged edible film dosage form of claim 52, wherein said at least one level of information comprises a barcode.

56. The packaged edible film dosage form of claim 52, wherein said indicia comprises a primary and a secondary level of information.

57. A method of indicating the presence of an active component in a film dosage form comprising the steps of:
(a) providing a film composition comprising at least one film-forming polymer, and at least one active component, said film composition having at least one surface;
(b) applying at least one indicia to said at least one surface of said film composition, wherein said at least one indicia corresponds to said at least one active present in said film composition thereby indicating the presence of said at least one active.

58. A method of applying indicia on an edible film composition comprising:
(a) providing a sheet of edible film composition comprising a film-forming polymer, and at least one active component, wherein said sheet has at least one surface; and
(b) applying indicia to said surface of said sheet of edible film composition, wherein said indicia comprises information corresponding to said edible film composition.

59. A method of providing information to a consumer with respect to an edible film product comprising,
(a) providing a sheet of edible film, said sheet having at least one surface for displaying indicia;
(b) applying indicia selected from the group consisting of a pattern, an image, a color, a shape or combination thereof, wherein said indicia provides information about the content or use of the product.

60. A method of providing information to a consumer with respect to a packaged edible film dosage form comprising:
(a) providing a sheet of edible film, said sheet having at least one surface for displaying indicia;
(b) dividing said sheet of edible film into individualized dosage forms;
(c) enclosing said individualized dosage forms in a sealed pouch;
(d) applying indicia selected from the group consisting of a barcode, a pattern, an image, a color, a shape or combination thereof, wherein said indicia provides information regarding the content or use of the product.

61. A method of testing a packaged edible film dosage form for quality control purposes comprising:
(a) irradiating a sample packaged edible film dosage form to establish a reference signal;
(b) measuring the value of a packaged edible film dosage form as it passes through a radiation device;
(c) comparing said value against the reference signal to create an output signal;
(e) using said output signal to classify said packaged edible film dosage form for quality control purposes.

62. A method of incorporating multiple levels of information into an edible film composition comprising:
(a) providing a film composition comprising at least one film-forming polymer, and at least one active component, said film composition having at least one surface;
(b) applying at least one indicia to said at least one surface of said film composition, wherein said at least one indicia provides a level of information selected from the group consisting of a primary level of information, a secondary level of information, a tertiary level of information, and combinations thereof.
